(19) United States
(12) Patent Application Publication
(10) Pub. No.: US 2018/0116520 A1
(43) Pub. Date: May 3, 2018

(54) IMAGING APPARATUS

(71) Applicant: OLYMPUS CORPORATION, Tokyo (JP)

(72) Inventor: Yusuke Yamamoto, Kawasaki-shi (JP)

(73) Assignee: OLYMPUS CORPORATION, Tokyo (JP)

(21) Appl. No.: 15/801,849

(22) Filed: Nov. 2, 2017

Related U.S. Application Data
(63) Continuation of application No. PCT/JP2015/067452, filed on Jun. 17, 2015.

(51) Int. Cl. A61B 5/00; A61B 1/04; A61B 1/00; A61B 1/06

(52) U.S. Cl. CPC A61B 5/0035; A61B 1/04; A61B 5/0071; A61B 5/0086; A61B 1/0002; A61B 1/00096; A61B 1/0646; A61B 1/0638; A61B 1/00009

(57) ABSTRACT

An imaging apparatus includes an imaging unit and a signal processing unit. The imaging unit generates a first image signal on the basis of visible light from an object and generates a second image signal on the basis of excitation light and fluorescence from the object. The signal processing unit generates a fluorescence image signal corresponding to the fluorescence on the basis of the first image signal and the second image signal. The signal processing unit determines a target area of the object on the basis of the first image signal. The signal processing unit determines a fluorescence area on the basis of the second image signal corresponding to the target area, and the fluorescence area generates the fluorescence in the object. The signal processing unit performs an emphasis process of the second image signal corresponding to the fluorescence area.

The present application is a continuation application based on International Patent Application No. PCT/JP2015/067452 filed Jun. 17, 2015, the content of which is incorporated herein by reference.

### BACKGROUND OF THE INVENTION

### Field of the Invention

The present invention relates to an imaging apparatus.

### Description of Related Art

Endoscope systems capable of special-light observation using infrared light in addition to ordinary observation using visible light are widely used. In such an endoscope system, a lesion found through the ordinary observation or the special-light observation can be treated using a treatment tool.

For example, in an endoscope system disclosed in Japanese Unexamined Patent Application, First Publication No. H10-201707, excitation light is emitted to a fluorescent material called indocyanine green (ICG), and fluorescence from a lesion is detected. ICG is administered to the inside of the body of an inspection target person in advance. ICG is excited in an infrared region by excitation light to emit fluorescence. The administered ICG is accumulated in a lesion of cancer or the like. Since strong fluorescence is generated from the lesion, an inspector can determine the presence/absence of a lesion on the basis of a captured fluorescence image.

In the endoscope system disclosed in Japanese Unexamined Patent Application, First Publication No. H10-201707, light including visible light and infrared light is emitted to an object. The wavelength band of the infrared light emitted to an object does not include the wavelength band of fluorescence but includes the wavelength band of the excitation light. Light reflected by an object and fluorescence (infrared fluorescence) generated from the object are imaged through a dichroic mirror or a dichroic prism built inside a camera head. A dividing means that devices visible light and fluorescence is provided, and accordingly, ordinary observation using visible light and special-light observation using infrared light can be simultaneously performed. In addition, fluorescence, red light, green light, and blue light are imaged using different image sensors through the dichroic mirror or the dichroic prism. For this reason, an image having high image quality can be acquired.

FIG. 9 shows the configuration of an endoscope apparatus 1001 similar to the configuration disclosed in Japanese Unexamined Patent Application, First Publication No. H10-201707. As shown in FIG. 9, the endoscope apparatus 1001 includes a light source unit 1010, an endoscope unit 1020, a camera head 1030, a processor 1040, and a monitor 1050. FIG. 9 shows schematic configurations of the light source unit 1010, the endoscope unit 1020, and the camera head 1030.

The light source unit 1010 includes a light source 1100, a band pass filter 1101, and a condenser lens 1102. The light source 1100 emits light of wavelengths ranging from the wavelength band of visible light to the wavelength band of infrared light. The wavelength band of infrared light includes the wavelength band of excitation light and the wavelength band of fluorescence. The wavelength band of fluorescence is a band having wavelengths longer than those of the wavelength band of excitation light in the wavelength band of infrared light. The band pass filter 1101 is disposed in the middle of an illumination optical path of the light source 1100. The band pass filter 1101 transmits only visible light and excitation light. The condenser lens 1102 condenses light transmitted through the band pass filter 1101. The wavelength band of the infrared light emitted by the light source 1100 may include at least the wavelength band of the excitation light.

FIG. 10 shows the transmission characteristic of the band pass filter 1101. In a graph shown in FIG. 10, the horizontal axis represents the wavelength, and the vertical axis represents the transmittance. The band pass filter 1101 transmits light of a wavelength band having wavelengths of about 370 nm to about 800 nm. On the other hand, the band pass filter 1101 blocks light of a wavelength band having wavelengths less than about 370 nm and light of a wavelength band having wavelengths of about 800 nm or more. The wavelength band of light transmitted by the band pass filter 1101 includes the wavelength band of visible light and the wavelength band of excitation light. The wavelength band of the excitation light is a band having wavelengths of about 750 nm to about 780 nm. The wavelength band of light blocked by the band pass filter 1101 includes the wavelength band of fluorescence. The wavelength band of fluorescence is a band having wavelengths of about 800 nm to about 900 nm.

The endoscope unit 1020 includes a light guide 1200, an illumination lens 1201, an objective lens 1202, and an image guide 1203. Light emitted from the light source 1100 is incident on the light guide 1200 through the band pass filter 1101 and the condenser lens 1102. The light guide 1200 transmits the light emitted from the light source 1100 to a tip end portion of the endoscope unit 1020. The light transmitted by the light guide 1200 is emitted to an object 1060 by the illumination lens 1201.

The objective lens 1202 is disposed to be adjacent to the illumination lens 1201 at the tip end portion of the endoscope unit 1020. The light reflected by the object 1060 and the fluorescence generated from the object 1060 are incident on the objective lens 1202. The light reflected by the object 1060 includes visible light and excitation light. In other words, light including the reflected light of the wavelength band of the visible light from the object 1060, the reflected light of the wavelength band of the excitation light, and the fluorescence emitted from the object 1060 is incident on the objective lens 1202. The objective lens 1202 forms an image of the light described above.

A front end face of the image guide 1203 is arranged at an image formation position of the objective lens 1202. The image guide 1203 transmits an optical image formed on the front end face to a rear end face.

The camera head 1030 includes an imaging lens 1300, a dichroic mirror 1301, an excitation light cutoff filter 1302, an image sensor 1303, a dichroic prism 1304, an image sensor 1305, an image sensor 1306, and an image sensor 1307. The imaging lens 1300 is arranged to face the rear end face of the image guide 1203. The imaging lens 1300 forms the optical image transmitted by the image guide 1203 at the image sensor 1303, the image sensor 1305, the image sensor 1306, and the image sensor 1307.

The dichroic mirror 1301 is arranged in an optical path from the imaging lens 1300 to the image formation position of the imaging lens 1300. Light passing through the imaging lens 1300 is incident on the dichroic mirror 1301. The dichroic mirror 1301 transmits visible light and reflects light other than the visible light. FIG. 11 shows the reflection and transmission characteristics of the dichroic mirror 1301. In a graph shown in FIG. 11, the horizontal axis represents the wavelength, and the vertical axis represents the transmittance. The dichroic mirror 1301 transmits light of a wavelength band having wavelengths less than about 700 nm. On the other hand, the dichroic mirror 1301 reflects light of a wavelength band having wavelengths of about 700 nm or more. The wavelength band of light transmitted by the dichroic mirror 1301 includes the wavelength band of visible light. In addition, the wavelength band of light reflected by the dichroic mirror 1301 includes the wavelength band of infrared light.

At the image formation position of light transmitted through the dichroic mirror 1301, an optical image of a visible light component is formed. On the other hand, at the image formation position of light reflected by the dichroic mirror 1301, an optical image of an infrared light component is formed.

The light reflected by the dichroic mirror 1301 is incident on the excitation light cutoff filter 1302. The light incident on the excitation light cutoff filter 1302 includes infrared light. The infrared light includes the excitation light and the fluorescence. The excitation light cutoff filter 1302 blocks the excitation light and transmits the fluorescence. FIG. 12 shows the transmission characteristics of the excitation light cutoff filter 1302. In a graph shown in FIG. 12, the horizontal axis indicates the wavelength, and the vertical axis indicates the transmittance. The excitation light cutoff filter 1302 blocks light of a wavelength band having wavelengths less than about 800 nm. On the other hand, the excitation light cutoff filter 1302 transmits light of a wavelength band having wavelengths of about 800 nm or more. The wavelength band of the light blocked by the excitation light cutoff filter 1302 includes the wavelength band of the excitation light. The wavelength band of the light transmitted by the excitation light cutoff filter 1302 includes the wavelength band of the fluorescence.

The fluorescence transmitted through the excitation light cutoff filter 1302 is incident on the image sensor 1303. The image sensor 1303 generates an IR signal on the basis of the fluorescence.

FIG. 13 shows the characteristics of ICG administered to the object 1060. In a graph shown in FIG. 13, the horizontal axis indicates the wavelength, and the vertical axis indicates the intensity. FIG. 13 shows the characteristics of excitation light exciting ICG and the characteristics of fluorescence emitted by ICG. The peak wavelength of the excitation light is about 770 nm, and the peak wavelength of the fluorescence is about 820 nm. Accordingly, when excitation light having wavelengths of about 750 nm to about 780 nm is emitted to the object 1060, fluorescence having wavelengths of about 800 nm to about 900 nm is generated from the object 1060. By detecting the fluorescence generated from the object 1060, the presence/absence of cancer can be detected. As shown in FIG. 10, the band pass filter 1101 transmits excitation light having wavelengths of about 750 nm to about 780 nm and blocks fluorescence having wavelengths of about 800 nm to about 900 nm. In addition, as shown in FIG. 12, the excitation light cutoff filter 1302 blocks excitation light having wavelengths of about 750 nm to about 780 nm.

The light of the wavelength band of visible light that has been transmitted through the dichroic mirror 1301 is incident on the dichroic prism 1304. The dichroic prism 1304 splits the light of the wavelength band of the visible light into light (red light) of a red wavelength band, light (green light) of a green wavelength band, and light (blue light) of a blue wavelength band. The red light passing through the dichroic prism 1304 is incident on the image sensor 1305. The image sensor 1305 generates an R signal on the basis of the red light. The green light passing through the dichroic prism 1304 is incident on the image sensor 1306. The image sensor 1306 generates a G signal on the basis of the green light. The blue light passing through the dichroic prism 1304 is incident on the image sensor 1307. The image sensor 1307 generates a B signal on the basis of the blue light.

The processor 1040 generates a visible light image signal on the basis of the R signal, the G signal, and the B signal and generates a fluorescence image signal on the basis of the IR signal. The monitor 1050 displays a visible light image on the basis of the visible light image signal and a fluorescence image on the basis of the fluorescence image signal. For example, the monitor 1050 displays the visible light image and the fluorescence image acquired at the same time to be aligned. Alternatively, the monitor 1050 displays the visible light image and the fluorescence image acquired at the same time to overlap each other.

In the endoscope apparatus 1001 shown in FIG. 9, the excitation light cutoff filter 1302 is arranged before the image sensor 1303 such that image sensor 1303 does not detect reflected light having the wavelength band of the excitation light that is reflected from the object 1060 but detects only the fluorescence out of light reflected by the dichroic mirror 1301. However, it is difficult to manufacture the excitation light cutoff filter 1302 that completely blocks light having the wavelength band of the excitation light. For this reason, the image sensor 1303 detects light of the fluorescence band and the remaining light having the wavelength band of the excitation light that cannot be blocked by the excitation light cutoff filter 1302.

FIG. 14 schematically shows the energy distribution of light incident on the image sensor 1303. In a graph shown in FIG. 14, the horizontal axis represents the wavelength, and the vertical axis represents the incident energy. As shown in FIG. 14, the wavelength band of light incident on the image sensor 1303 includes the wavelength band of the excitation light having wavelengths of about 700 nm to about 800 nm and the fluorescence band having wavelengths of about 800 nm to about 900 nm. In other words, fluorescence emitted from the object 1060 and a part of light of the wavelength band of the excitation light that cannot be blocked by the excitation light cutoff filter 1302 are incident on the image sensor 1303.

The fluorescence emitted from the object 1060 is weaker than the excitation light. For this reason, when a part of light of the wavelength band of the excitation light that cannot be blocked by the excitation light cutoff filter 1302 is incident on the image sensor 1303, there are cases in which the signal value of an IR signal generated by a first pixel of the image sensor 1303 is larger than the signal value of an IR signal generated by a second pixel of the image sensor 1303. Here, the first pixel is a pixel on which light from an object not emitting fluorescence and having high reflectivity of the excitation light is incident. The second pixel is a pixel on which light from an object emitting fluorescence and having high reflectivity of the excitation light is incident. For this reason, there are cases in which the signal value of an IR signal generated by a pixel of the image sensor 1303, on which light from an area of the object 1060 not emitting fluorescence is incident, is large. As a result, there are cases in which the area of the object 1060 not emitting fluorescence is displayed brightly in a fluorescence image.

When the excitation light transmitted through the excitation light cutoff filter 1302 is uniformly incident on the light receiving face of the image sensor 1303, a signal component generated in each pixel of the image sensor 1303 on the basis of the excitation light is uniform. For this reason, by subtracting an offset component based on the excitation light from an IR signal generated in each pixel of the image sensor 1303, the processor 1040 can calculate an IR signal that is based on only the fluorescence.

### SUMMARY OF INVENTION

According to a first aspect of the present invention, an imaging apparatus includes an imaging unit and a signal processing unit. The imaging unit generates a first image signal on the basis of visible light from an object and generates a second image signal on the basis of excitation light and fluorescence from the object. The signal processing unit generates a fluorescence image signal corresponding to the fluorescence on the basis of the first image signal and the second image signal. The signal processing unit determines a target area of the object on the basis of the first image signal. The signal processing unit determines a fluorescence area on the basis of the second image signal corresponding to the target area, and the fluorescence area generates the fluorescence in the object. The signal processing unit performs an emphasis process of the second image signal corresponding to the fluorescence area.

According to a second aspect of the present invention, in the first aspect, the signal processing unit may perform the emphasis process by performing addition or multiplication of a predetermined value only for a signal value of the second image signal corresponding to the fluorescence area.

According to a third aspect of the present invention, in the first aspect, the signal processing unit may perform the emphasis process by performing addition or multiplication of a value according to a signal value of the second image signal corresponding to the fluorescence area only for the signal value.

According to a fourth aspect of the present invention, in the first aspect, the signal processing unit may calculate an area determination coefficient of each pixel according to a degree of correlation between a signal value of the first image signal of each pixel and a reference value. The reference value corresponds to a value expected as a signal value of the first image signal corresponding to the target area. The signal processing unit may determine the target area on the basis of the area determination coefficient.

According to a fifth aspect of the present invention, in the fourth aspect, the signal processing unit may multiply a signal value of the second image signal of each pixel for which the emphasis process is performed by the area determination coefficient of each pixel.

According to a sixth aspect of the present invention, in the first aspect, the imaging unit may include a dichroic mirror, a visible light imaging unit, an excitation light cutoff filter, and a fluorescence imaging unit. The dichroic mirror splits first light from the object into second light and third light. The first light includes the visible light, the excitation light, and the fluorescence. The second light includes the visible light. The third light includes the excitation light and the fluorescence. The visible light imaging unit, on which the second light is incident, generates the first image signal. The excitation light cutoff filter, on which the third light is incident, has first transmittance for the fluorescence and second transmittance for the excitation light. The first transmittance is higher than the second transmittance. The fluorescence imaging unit, on which the third light transmitted through the excitation light cutoff filter is incident, generates the second image signal. The visible light imaging unit and the fluorescence imaging unit may be connected to the signal processing unit.

According to a seventh aspect of the present invention, in the first aspect, the signal processing unit may include a memory and a target area determining unit. Object characteristic information representing characteristics of the object is recorded in the memory. The object characteristic information is generated on the basis of the first image signal of the object. The target area determining unit determines the target area on the basis of the object characteristic information recorded in the memory and the first image signal.

According to an eighth aspect of the present invention, in the first aspect, the signal processing unit may calculate saturation and hue of each pixel on the basis of a signal value of the first image signal of each pixel. The signal processing unit may determine the target area on the basis of the saturation and the hue of each pixel.

### BRIEF DESCRIPTION OF THE DRAWINGS

FIG. 1 is a block diagram showing the configuration of an endoscope apparatus according to a first embodiment of the present invention.

FIG. 2 is a reference diagram showing the concept of determining a target area according to the first embodiment of the present invention.

FIG. 3 is a reference diagram showing the concept of determining a fluorescence area according to the first embodiment of the present invention.

FIG. 4 is a reference diagram showing the concept of determining a fluorescence area according to the first embodiment of the present invention.

FIG. 5 is a block diagram showing the configuration of an endoscope apparatus according to a first modified example of the first and second embodiments of the present invention.

FIG. 6 is a graph showing the characteristics of an excitation light cutoff filter according to the first modified example of the first and second embodiments of the present invention.

FIG. 7 is a reference diagram showing the pixel arrangement of an image sensor according to the first modified example of the first and second embodiments of the present invention.

FIG. 8 is a block diagram showing the configuration of an endoscope apparatus according to a second modified example of the first and second embodiments of the present invention.

FIG. 9 is a block diagram showing the configuration of an endoscope apparatus of a conventional technology.

FIG. 10 is a graph showing the characteristics of a band pass filter.

FIG. 11 is a graph showing the characteristics of a dichroic mirror.

FIG. 12 is a graph showing the characteristics of an excitation light cutoff filter.

FIG. 13 is a graph showing the characteristics of indocyanine green (ICG).

FIG. 14 is a graph showing an energy distribution of light incident on an image sensor.

### DETAILED DESCRIPTION OF THE INVENTION

Embodiments of the present invention will be described with reference to the drawings. In each embodiment described below, an endoscope apparatus that is an example of an imaging apparatus will be described. The present invention can be applied to an apparatus, a system, a module, and the like having an imaging function.

### First Embodiment

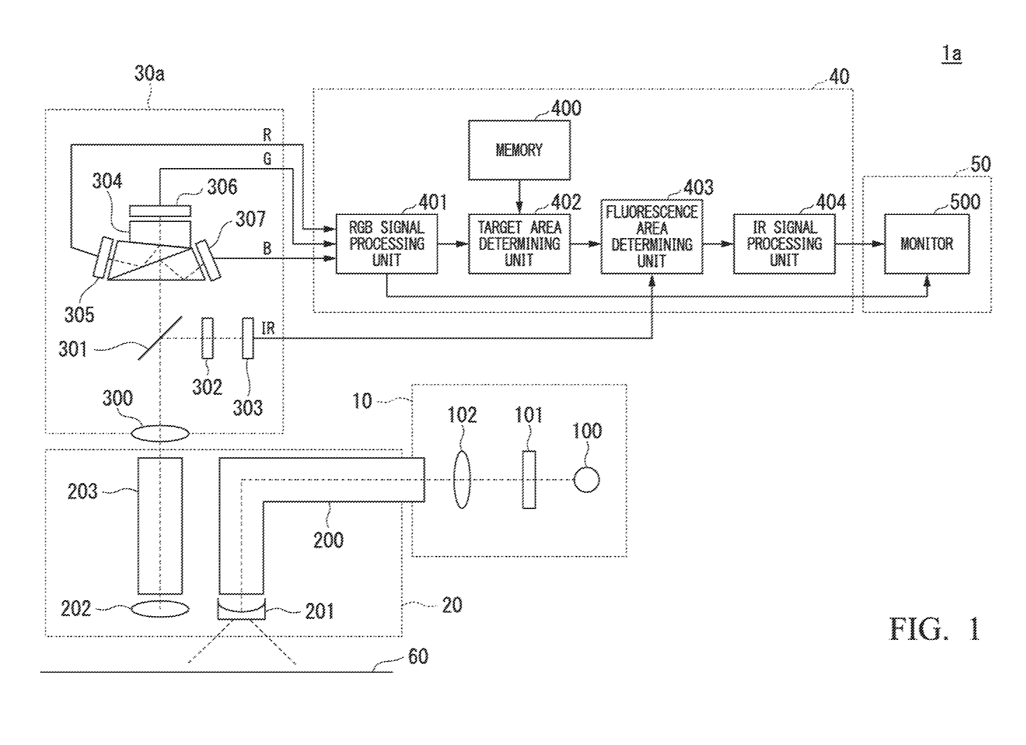

FIG. 1 shows the configuration of an endoscope apparatus 1a according to a first embodiment of the present invention. As shown in FIG. 1, the endoscope apparatus 1a includes a light source unit 10, an endoscope unit 20, a camera head 30a (imaging unit), a signal processing unit 40, and a display unit 50. FIG. 1 shows schematic configurations of the light source unit 10, the endoscope unit 20, and the camera head 30a.

The light source unit 10 includes a light source 100, a band pass filter 101, and a condenser lens 102. The light source 100 emits light of the wavelengths ranging from the wavelength band of visible light to the wavelength band of infrared light. The wavelength band of visible light includes a red wavelength band, a green wavelength band, and a blue wavelength band. The red wavelength band is a band having longer wavelengths than the green wavelength band. The green wavelength band is a band having longer wavelengths than the blue wavelength band. The wavelength band of infrared light is a band having longer wavelengths than the red wavelength band. The wavelength band of infrared light includes the wavelength band of excitation light and the wavelength band of fluorescence. The wavelength band of fluorescence is a band having longer wavelengths than the wavelength band of excitation light. In other words, the wavelengths of infrared light are longer than the wavelengths of red light. The wavelengths of red light are longer than the wavelengths of green light. The wavelengths of green light are longer than the wavelengths of blue light. The wavelength band of infrared light emitted by the light source 100 may include at least the wavelength band of excitation light.

The band pass filter 101 is disposed in the middle of an illumination optical path of the light source 100. The band pass filter 101 transmits only visible light and excitation light. The condenser lens 102 condenses light transmitted through the band pass filter 101.

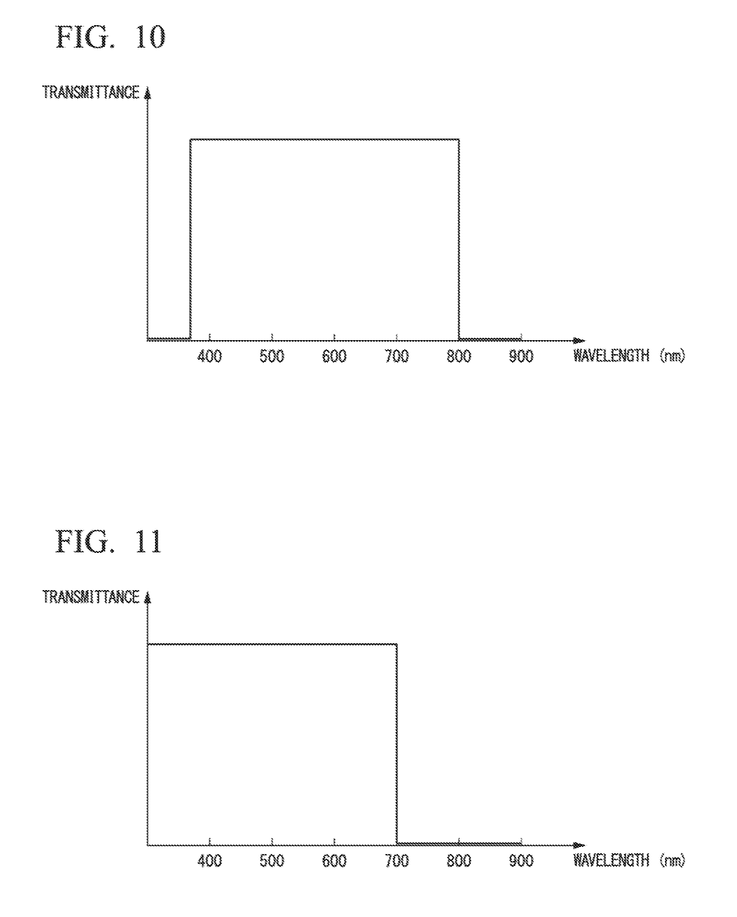

The transmission characteristics of the band pass filter 101 are similar to transmission characteristics shown in FIG. 10. The band pass filter 101 transmits light of a wavelength band having wavelengths of about 370 nm to about 800 nm. On the other hand, the band pass filter 101 blocks light of a wavelength band having wavelengths less than about 370 nm and light of a wavelength band having wavelengths of about 800 nm or more. The wavelength band of light transmitted by the band pass filter 101 includes the wavelength band of visible light and the wavelength band of excitation light. The wavelength band of excitation light is a band having wavelengths of about 750 nm to about 780 nm. The wavelength band of light blocked by the band pass filter 101 includes the wavelength band of fluorescence. The wavelength band of fluorescence is a band having wavelengths of about 800 nm to about 900 nm.

The endoscope unit 20 includes a light guide 200, an illumination lens 201, an objective lens 202, and an image guide 203. Light emitted from the light source 100 is incident on the light guide 200 through the band pass filter 101 and the condenser lens 102. The light guide 200 transmits the light emitted from the light source 100 to a tip end portion of the endoscope unit 20. The light transmitted by the light guide 200 is emitted to an object 60 through the illumination lens 201.

The objective lens 202 is disposed to be adjacent to the illumination lens 201 in the tip end portion of the endoscope unit 20. Light reflected by the object 60 and fluorescence generated from the object 60 are incident on the objective lens 202. The light reflected by the object 60 includes visible light and excitation light. In other words, light including the reflected light of the wavelength band of the visible light from the object 60, the reflected light of the wavelength band of the excitation light, and the fluorescence emitted from the object 60 is incident on the objective lens 202. The objective lens 202 forms an image of the light described above.

A front end face of the image guide 203 is arranged at an image formation position of the objective lens 202. The image guide 203 transmits an optical image formed on the front end face to a rear end face.

The camera head 30a includes an imaging lens 300, a dichroic mirror 301, an excitation light cutoff filter 302, an image sensor 303 (fluorescence imaging unit), a dichroic prism 304, an image sensor 305 (visible light imaging unit), an image sensor 306 (visible light imaging unit), and an image sensor 307 (visible light imaging unit). The imaging lens 300 is arranged to face the rear end face of the image guide 203. The imaging lens 300 forms the optical image transmitted by the image guide 203 at the image sensor 303, the image sensor 305, the image sensor 306, and the image sensor 307.

First light from the object 60 includes second light and third light. The second light includes visible light. The visible light includes red light, green light, and blue light. Third light includes excitation light and fluorescence. The wavelengths of the fluorescence are longer than the wavelengths of the excitation light.

The dichroic mirror 301 is arranged in an optical path from the imaging lens 300 to the image formation position of the imaging lens 300. The first light passing through the imaging lens 300, in other words, the first light from the object 60, is incident on the dichroic mirror 301. The dichroic mirror 301 transmits visible light and reflects light other than the visible light. The reflection and transmission characteristics of the dichroic mirror 301 are similar to the reflection and transmission characteristics of a dichroic mirror 1301 shown in FIG. 11. The dichroic mirror 301 transmits light of a wavelength band having wavelengths less than about 700 nm. On the other hand, the dichroic mirror 301 reflects light of a wavelength band having wavelengths of about 700 nm or more. The wavelength band of light transmitted by the dichroic mirror 301 includes the wavelength band of visible light. In addition, the wavelength band of light reflected by the dichroic mirror 301 includes the wavelength band of infrared light. In other words, the dichroic mirror 301 transmits the second light and reflects the third light. In this way, the dichroic mirror 301 splits the first light from the object 60 into the second light and the third light.

At the image formation position of light transmitted through the dichroic mirror 301, an optical image of a visible light component is formed. On the other hand, at the image formation position of light reflected by the dichroic mirror 301, an optical image of an infrared light component is formed.

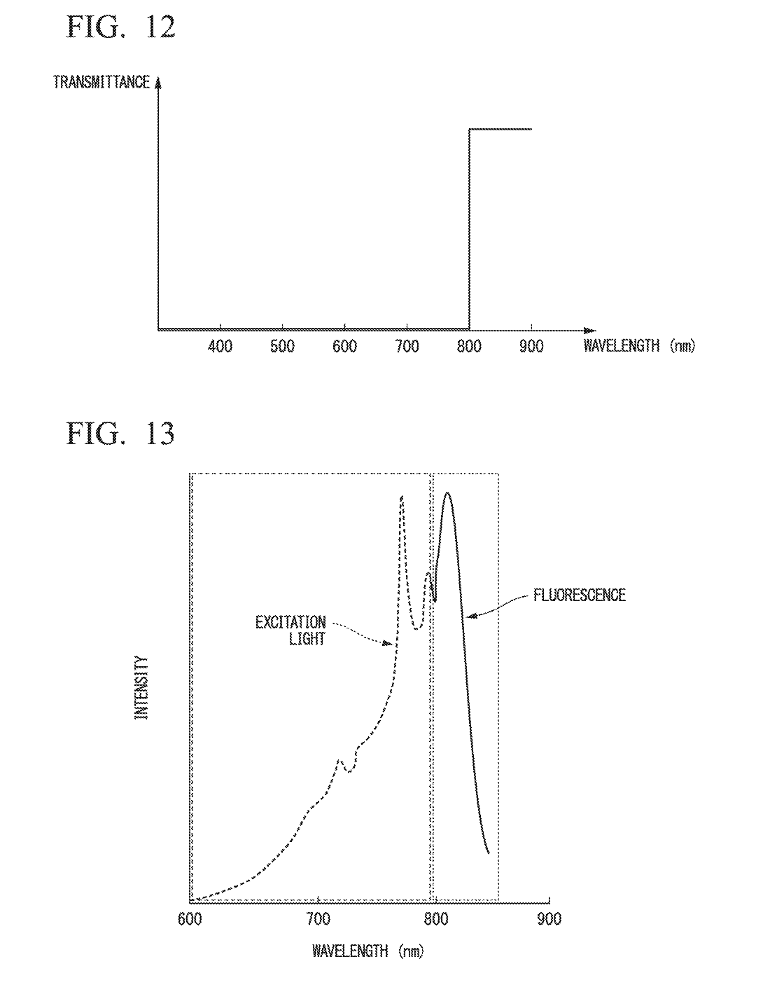

The third light reflected by the dichroic mirror 301 is incident on the excitation light cutoff filter 302. The light incident on the excitation light cutoff filter 302 includes infrared light. The infrared light includes the excitation light and the fluorescence. The excitation light cutoff filter 302 blocks the excitation light and transmits the fluorescence. The transmission characteristics of the excitation light cutoff filter 302 are similar to the transmission characteristics of an excitation light cutoff filter 1302 shown in FIG. 12. The excitation light cutoff filter 302 blocks light of a wavelength band having wavelengths less than about 800 nm. On the other hand, the excitation light cutoff filter 302 transmits light of a wavelength band having wavelengths of about 800 nm or more. The wavelength band of light blocked by the excitation light cutoff filter 302 includes the wavelength band of the excitation light. The wavelength band of light transmitted by the excitation light cutoff filter 302 includes the wavelength band of the fluorescence. The blocking characteristics of the excitation light cutoff filter 302 for the excitation light are not perfect. The excitation light cutoff filter 302 blocks a part of light of the wavelength band of the excitation light and transmits the remaining light of the wavelength band of the excitation light and the fluorescence.

The part of the light of the wavelength band of the excitation light and the fluorescence that have been transmitted through the excitation light cutoff filter 302 are incident on the image sensor 303. The image sensor 303 generates an IR signal (second image signal) on the basis of the excitation light and the fluorescence that have been transmitted through the excitation light cutoff filter 302.

The second light that has been transmitted through the dichroic mirror 301 is incident on the dichroic prism 304. The dichroic prism 304 splits the second light into light (red light) of the red wavelength band, light (green light) of the green wavelength band, and light (blue light) of the blue wavelength band. The red light passing through the dichroic prism 304 is incident on the image sensor 305. The image sensor 305 generates an R signal (first image signal) on the basis of the red light. The green light passing through the dichroic prism 304 is incident on the image sensor 306. The image sensor 306 generates a G signal (first image signal) on the basis of the green light. The blue light passing through the dichroic prism 304 is incident on the image sensor 307. The image sensor 307 generates a B signal (first image signal) on the basis of the blue light.

The R signal includes signal values (pixel values) of a plurality of pixels arranged in the image sensor 305. The G signal includes signal values (pixel values) of a plurality of pixels arranged in the image sensor 306. The B signal includes signal values (pixel values) of a plurality of pixels arranged in the image sensor 307. The IR signal includes signal values (pixel values) of a plurality of pixels arranged in the image sensor 303.

As described above, the camera head 30a (imaging unit) includes the dichroic mirror 301, the excitation light cutoff filter 302, the image sensor 305 (visible light imaging unit), the image sensor 306 (visible light imaging unit), the image sensor 307 (visible light imaging unit), and the image sensor 303 (fluorescence imaging unit). The dichroic mirror 301 splits the first light from the object 60 into the second light and the third light. The first light includes visible light, excitation light, and fluorescence. The second light includes visible light. The third light includes excitation light and fluorescence. The second light is incident on the image sensor 305, the image sensor 306, and the image sensor 307. The image sensor 305, the image sensor 306, and the image sensor 307 generate signals (first image signals) on the basis of visible light. The transmittance of the excitation light cutoff filter 302 for fluorescence is higher than the transmittance of the excitation light cutoff filter 302 for excitation light. The third light is incident on the excitation light cutoff filter 302. The third light that has been transmitted through the excitation light cutoff filter 302 is incident on the image sensor 303. The image sensor 303 generates an IR signal (second image signal) on the basis of the excitation light and the fluorescence. The image sensor 305, the image sensor 306, the image sensor 307, and the image sensor 303 are connected to the signal processing unit 40.

The signal processing unit 40 generates a visible light image signal on the basis of the R signal, the G signal, and the B signal. The visible light image signal is a signal used for displaying a visible light image. In addition, the signal processing unit 40 generates a fluorescence image signal on the basis of at least one of the R signal, the G signal, and the B signal, and the IR signal. The fluorescence image signal is a signal used for displaying a fluorescence image.

The display unit 50 includes a monitor 500. The monitor 500 displays a visible light image on the basis of the visible light image signal and a fluorescence image on the basis of the fluorescence image signal. For example, the monitor 500 displays the visible light image and the fluorescence image acquired at the same time in alignment. Alternatively, the monitor 500 displays the visible light image and the fluorescence image acquired at the same time in an overlapping manner.

As described above, the endoscope apparatus 1a (imaging apparatus) includes the camera head 30a (imaging unit) and the signal processing unit 40. The camera head 30a generates first image signals (an R signal, a G signal, and a B signal) on the basis of the visible light from the object 60. The camera head 30a generates a second image signal (IR signal) on the basis of the excitation light and the fluorescence from the object 60. The signal processing unit 40 generates a fluorescence image signal corresponding to the fluorescence on the basis of the first image signals and the second image signal. The signal processing unit 40 determines a target area of the object 60 on the basis of the first image signals. The signal processing unit 40 determines a fluorescence area on the basis of the second image signal corresponding to the target area. The fluorescence area generates fluorescence in the object 60. The signal processing unit 40 performs an emphasis process of the second image signal corresponding to the fluorescence area. For this reason, the endoscope apparatus 1a can generate a fluorescence image signal for displaying a fluorescence image in which the fluorescence area is brightened more clearly.

A detailed configuration of the signal processing unit 40 will be described. The signal processing unit 40 includes a memory 400, an RGB signal processing unit 401, a target area determining unit 402, a fluorescence area determining unit 403, and an IR signal processing unit 404. For example, the memory 400 is a volatile or nonvolatile recording medium. For example, the RGB signal processing unit 401, the target area determining unit 402, the fluorescence area determining unit 403, and the IR signal processing unit 404 are mounted as processors. Alternatively, the RGB signal processing unit 401, the target area determining unit 402, the fluorescence area determining unit 403, and the IR signal processing unit 404 are mounted as hardware such as an application specific integrated circuit (ASIC) or the like.

Object characteristic information representing the characteristics of the object 60 is recorded in the memory 400. In other words, the memory 400 stores the object characteristic information. The object characteristic information is generated on the basis of the first image signals (the R signal, the G signal, and the B signal) of the object 60. For example, the object characteristic information is RGB information representing the spectral reflection characteristics of the object 60 for visible light.

The RGB signal processing unit 401 generates RGB information of each pixel on the basis of the first image signals (the R signal, the G signal, and the B signal). The RGB information generated by the RGB signal processing unit 401 is output to the target area determining unit 402.

The target area determining unit 402 determines a target area of the object 60 on the basis of the first image signals (the R signal, the G signal, and the B signal). In other words, the target area determining unit 402 determines a target area of the object 60 on the basis of the object characteristic information (RGB information) recorded in the memory 400 and the RGB information generated by the RGB signal processing unit 401. Target area information representing the target area is output to the fluorescence area determining unit 403. The target area information includes positional information of pixels corresponding to the target area.

The fluorescence area determining unit 403 determines a fluorescence area on the basis of the second image signal (IR signal) corresponding to the target area. In other words, the fluorescence area determining unit 403 determines a fluorescence area on the basis of the second image signals of pixels represented by the target area information. Fluorescence area information representing the fluorescence area is output to the IR signal processing unit 404. The fluorescence area information includes the positional information of pixels corresponding to the fluorescence area.

The IR signal processing unit 404 performs an emphasis process of the second image signal (IR signal) corresponding to the fluorescence area. In other words, the IR signal processing unit 404 performs an emphasis process of the second image signals of pixels represented by the fluorescence area information. The IR signal processing unit 404 performs an emphasis process of the second image signals such that signal values of the pixels corresponding to the fluorescence area are larger than the signal values of pixels corresponding to an area other than the fluorescence area in the second image signal.

The detail of the RGB information that is object characteristic information recorded in the memory 400 will be described. For example, the object 60 that is an observation target of the endoscope apparatus 1a is an organ of a human body. For example, the object 60 is the large intestine, the small intestine, the stomach, or the liver. After ICG is injected into a vein of a test subject, the administered ICG flows through blood vessels and lymphatic vessels. Accordingly, target areas in fluorescence observation using ICG are blood vessels and lymphatic vessels. The spectral reflection characteristics of a target area such as a blood vessel or a lymphatic vessel for visible light are different from the spectral reflection characteristics of other areas (for example, fat or the like) of the observation target for visible light. For this reason, by analyzing the R signal, the G signal, and the B signal, the target area of the imaged object 60 can be detected.

For example, the RGB information is a ratio between signal values of the R signal, the G signal, and the B signal. In other words, the RGB information includes a ratio between the signal values of the R signal and the G signal and a ratio between the signal values of the R signal and the B signal. For example, the ratio between the signal values of the R signal and the G signal in a target area is in the range of X1 to X2. Here, X2 is larger than X1. For example, the ratio between the signal values of the R signal and the B signal in a target area is in the range of Y1 to Y2. Here, Y2 is larger than Y1. The range of X1 to X2 and the range of Y1 to Y2 are recorded in the memory 400 as the RGB information.

The RGB information may be the saturation and the hue. The saturation is an index representing the vividness of a color. The saturation of achromatic colors (black, white, and gray) is “0.” As the color becomes vivid, the saturation increases. In other words, the saturation of a more vivid color is larger. The hue is an index representing the phase of a color such as red, yellow, green, blue, or violet. The numerical value of the hue is different for each phase of the color. RGB signals can be converted into pixel values (hue, saturation, and luminance) of an HIS color space defined by three elements of hue (H), saturation (S), and luminance (I). The range of each of the saturation and the hue is recorded in the memory 400.

A detailed operation of the signal processing unit 40 will be described. An R signal output from the image sensor 305, a G signal output from the image sensor 306, a B signal output from the image sensor 307, and an IR signal output from the image sensor 303 are input to the signal processing unit 40. The R signal, the G signal, and the B signal are input to the RGB signal processing unit 401. The IR signal is input to the fluorescence area determining unit 403. The pixels of the image sensor 305, the image sensor 306, the image sensor 307, and the image sensor 303 correspond to each other. For example, the numbers of pixels of the image sensor 305, the image sensor 306, the image sensor 307, and the image sensor 303 are the same.

The signal processing unit 40 (RGB signal processing unit 401) generates RGB information of each pixel on the basis of the R signal, the G signal, and the B signal. When the RGB information is generated, the signal processing unit 40 (RGB signal processing unit 401) performs the following process. The signal processing unit 40 (RGB signal processing unit 401) generates RGB information of a pixel on the basis of an R signal, a G signal, and a B signal of the pixel corresponding to each other. When the RGB information is ratios between the signal values of an R signal, a G signal, and a B signal, the signal processing unit 40 (RGB signal processing unit 401) calculates a ratio between the signal values of the R signal and the G signal and a ratio between the signal values of the R signal and the B signal. The signal processing unit 40 (RGB signal processing unit 401) outputs the RGB information including the calculated ratios to the target area determining unit 402.

When the RGB information is the saturation and the hue, the signal processing unit 40 (RGB signal processing unit 401) calculates the saturation and the hue of each pixel on the basis of the signal values of each pixel of the first image signals (the R signal, the G signal, and the B signal). The signal processing unit 40 (RGB signal processing unit 401) outputs the RGB information including the saturation and the hue that have been calculated to the target area determining unit 402.

In addition, the signal processing unit 40 (RGB signal processing unit 401) generates a visible light image signal on the basis of the R signal, the G signal, and the B signal. The signal processing unit 40 (RGB signal processing unit 401) may perform image processing such as an interpolation process for at least one of the R signal, the G signal, and the B signal. The signal processing unit 40 (RGB signal processing unit 401) outputs the visible light image signal to the monitor 500.

The RGB information generated by the signal processing unit 40 (RGB signal processing unit 401) may be recorded in the memory 400. For example, an object 60 including a known target area is imaged, and an R signal, a G signal, and a B signal are generated. In addition, a visible light image based on the visible light image signal of the object 60 including a known target area is displayed on the monitor 500. A target area is designated by an observer on the basis of this visible light image. The signal processing unit 40 (RGB signal processing unit 401) generates RGB information on the basis of an R signal, a G signal, and a B signal corresponding to the target area designated by the observer.

For example, the signal processing unit 40 (RGB signal processing unit 401) calculates a ratio between the signal values of the R signal and the (signal of each pixel of the target area and a ratio between the signal values of the R signal and the B signal of each pixel. A minimum value X1 and a maximum value X2 of the ratio between the signal values of the R signal and the G signal of each pixel of the target area are recorded in the memory 400 as RGB information. In addition, a minimum value Y1 and a maximum value Y2 of the ratio between the signal values of the B signal and the G signal of each pixel of the target area are recorded in the memory 400 as RGB information.

Alternatively, the signal processing unit 40 (RGB signal processing unit 401) calculates the saturation and the hue of each pixel of the target area. The range of each of the saturation and the hue of the target area is recorded in the memory 400 as RGB information.

The signal processing unit 40 (the target area determining unit 402) determines a target area of the object 60 on the basis of the object characteristic information (RGB information) recorded in the memory 400 and the first image signals (the R signal, the G signal, and the B signal). When a target area is determined, the signal processing unit 40 (the target area determining unit 402) performs the following process. The signal processing unit 40 (the target area determining unit 402) reads the RGB information from the memory 400. The signal processing unit 40 (the target area determining unit 402) compares the RGB information recorded in the memory 400 with the RGB information generated by the RGB signal processing unit 401. The signal processing unit 40 (the target area determining unit 402) determines a target area of the object 60 on the basis of a result of the comparison.

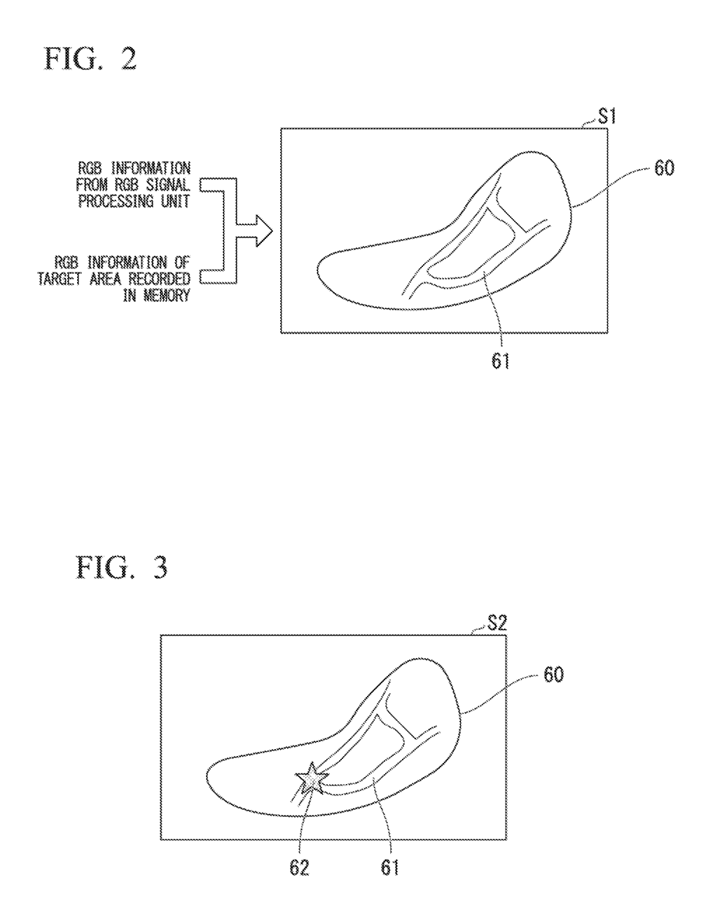

FIG. 2 shows the concept of determining a target area. An imaging area S1 is one imaging area of any one of the image sensor 305, the image sensor 306, and the image sensor 307. In the imaging area S1, an image of an object 60 based on any one of the red light, the green light, and the blue light is formed. The object 60 includes a target area 61. The signal processing unit 40 (the target area determining unit 402) compares the RGB information recorded in the memory 400 with the RGB information generated by the RGB signal processing unit 401 for each pixel. Accordingly, the signal processing unit 40 (the target area determining unit 402) determines whether or not each pixel is included in the target area 61.

When the RGB information is a ratio between the signal values of the R signal, the G signal, and the B signal, the signal processing unit 40 (the target area determining unit 402) determines whether or not the ratio calculated by the RGB signal processing unit 401 is included in the range of ratios recorded in the memory 400. For example, the signal processing unit 40 (the target area determining unit 402) determines whether or not a ratio Prg between the signal values of the R signal and the G signal calculated by the RGB signal processing unit 401 is included in the range of a ratio between the signal values of the R signal and the G signal recorded in the memory 400. The range of the ratio between the signal values of the R signal and the G signal is X1 to X2.

Similarly, the signal processing unit 40 (the target area determining unit 402) determines whether or not a ratio Prb between the signal values of the R signal and the 13 signal calculated by the RGB signal processing unit 401 is included in the range of a ratio between the signal values of the R signal and the 13 signal recorded in the memory 400. The range of the ratio between the signal values of the R signal and the B signal is Y1 to Y2. When the ratio Prg is X1 or more and less than X2, and the ratio Prb is Y1 or more and less than Y2, the signal processing unit 40 (the target area determining unit 402) determines that a pixel that is the determination target is included in the target area. On the other hand, when the ratio Prg is less than X1 or more than X2, the signal processing unit 40 (the target area determining unit 402) determines that the pixel that is the determination target is not included in the target area. Also, when the ratio Prb is less than Y1 or more than Y2, the signal processing unit 40 (the target area determining unit 402) determines that the pixel that is the determination target is not included in the target area.

When the RGB information is the saturation and the hue, the signal processing unit 40 (the target area determining unit 402) determines a target area on the basis of the saturation and the hue of each pixel of the first image signals (the R signal, the G signal, and the B signal). In other words, the signal processing unit 40 (the target area determining unit 402) determines whether or not the saturation Ps calculated by the RGB signal processing unit 401 is included in the range of the saturation Psm recorded in the memory 400. Similarly, the signal processing unit 40 (the target area determining unit 402) determines whether or not the hue Ph calculated by the RGB signal processing unit 401 is included in the range of the hue Phm recorded in the memory 400.

When the saturation Ps is included in the range of the saturation Psm, and the hue Ph is in the range of the hue Phm, the signal processing unit 40 (the target area determining unit 402) determines that the pixel that is the determination target is included in the target area. On the other hand, when the saturation Ps is not included in the range of the saturation Psm, the signal processing unit 40 (the target area determining unit 402) determines that the pixel that is the determination target is not included in the target area. Also, when the hue Ph is not included in the range of the hue Phm, the signal processing unit 40 (the target area determining unit 402) determines that the pixel that is the determination target is not included in the target area.

The signal processing unit 40 (the target area determining unit 402) generates target area information on the basis of a result of the determination of the target area. The target area information includes the positional information of the pixel determined to be included in the target area. The signal processing unit 40 (the target area determining unit 402) outputs target area information to the fluorescence area determining unit 403.

The signal processing unit 40 (the fluorescence area determining unit 403) determines a fluorescence area on the basis of the signal value of each pixel of the second image signal (IR signal) corresponding to the target area. When a fluorescence area is determined, the signal processing unit 40 (the fluorescence area determining unit 403) performs the following process. The signal processing unit 40 (the fluorescence area determining unit 403) compares the signal value of the IR signal of each pixel represented by the target area information with a reference value α. The signal processing unit 40 (the fluorescence area determining unit 403) determines a fluorescence area of the target area of the object 60 on the basis of a result of the comparison.

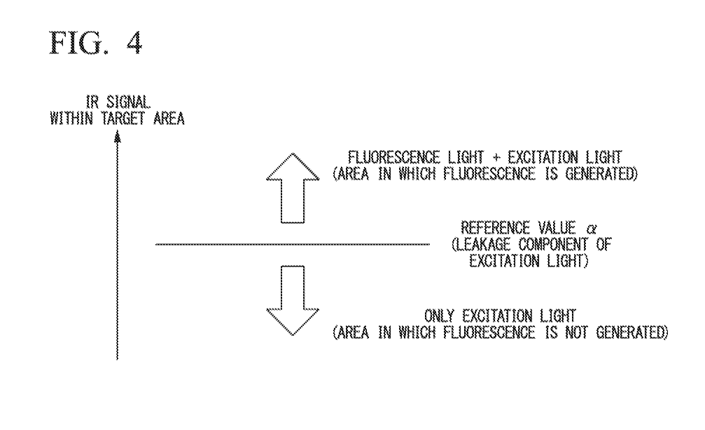

FIGS. 3 and 4 show the concept of determining a fluorescence area. An imaging area S2 is an imaging area of the image sensor 303. An image of the object 60 based on the excitation light and the fluorescence is formed in the imaging area S2. The object 60 includes a target area 61.

ICG flows through blood vessels and lymphatic vessels. However, ICG does not necessarily flows through all the blood vessels and lymphatic vessels inside the object 60. For this reason, the signal processing unit 40 (the fluorescence area determining unit 403) determines an area of the target area 61 in which ICG emits light and an area of the target area 61 in which ICG does not emit light.

In an area of a lesion, the administered ICG is accumulated, and fluorescence is generated. For this reason, in an area of a lesion, the signal value of the IR signal is larger than that of an area of no lesion. In other words, an IR signal corresponding to an area of a lesion of the target area includes signal components based on the fluorescence and a part of the excitation light. For this reason, the signal value of an IR signal corresponding to an area of a lesion is large. On the other hand, an IR signal corresponding to an area of the target area with no lesion includes a signal component based on only a part of the excitation light. For this reason, the signal value of an IR signal corresponding to an area with no lesion is small.

The signal processing unit 40 (the fluorescence area determining unit 403) compares the signal value of the IR signal with the reference value α for each pixel of the target area. Accordingly, the signal processing unit 40 (the fluorescence area determining unit 403) determines whether or not each pixel of the target area is included in the fluorescence area. The reference value α is a signal value based on excitation light that has been transmitted through the excitation light cutoff filter 302, in other words, a signal value based on a leakage component of the excitation light.

When the signal value of the IR signal of the pixel of the target area is the reference value α or more, the signal processing unit 40 (the fluorescence area determining unit 403) determines that the pixel that is the determination target is included in the fluorescence area. On the other hand, when the signal value of the IR signal of the pixel of the target area is less than the reference value α, the signal processing unit 40 (the fluorescence area determining unit 403) determines that the pixel that is the determination target is not included in the fluorescence area.

For example, the reference value α is determined as below. An object 60 including a known target area is imaged, and an R signal, a G signal, and a B signal are generated. In addition, a visible light image based on a visible light image signal of the object 60 including the known target area is displayed on the monitor 500. A target area is designated by an observer on the basis of this visible light image. The signal processing unit 40 (the RGB signal processing unit 401) calculates the reflectivity of the excitation light on the basis of the R signal, the (i signal, and the B signal corresponding to the target area designated by the observer. The signal processing unit 40 (the RGB signal processing unit 401) calculates the reflectivity of the target area for the excitation light for each type of the object 60. For example, the types of the object 60 are the large intestine, the small intestine, the stomach, and the liver. The reflectivity of each type of the object 60 for the excitation light is recorded in the memory 400.

When an object 60 that is the observation target is observed, the signal processing unit 40 (the RGB signal processing unit 401) reads the reflectivity of the excitation light corresponding to the type of the object 60 from the memory 400. The signal processing unit 40 (the RGB signal processing unit 401) calculates a reflected light intensity of the excitation light in the target area on the basis of the intensity of the light source 100 and the reflectivity of the excitation light. The calculated reflected light intensity is the reference value α.

The signal processing unit 40 (the fluorescence area determining unit 403) may determine a fluorescence area by comparing the IR signals of pixels of the target area. For example, when a value acquired by subtracting the signal value of the IR signal of the second pixel of the target area from the signal value of the IR signal of the first pixel of the target area is a reference value β or more, the signal processing unit 40 (the fluorescence area determining unit 403) determines that the first pixel is included in the fluorescence area. On the other hand, when the value acquired by subtracting the signal value of the IR signal of a second pixel of the target area from the signal value of the IR signal of a first pixel of the target area is less than the reference value β, the signal processing unit 40 (the fluorescence area determining unit 403) determines that the first pixel is not included in the fluorescence area. For example, the second pixel is a pixel of which the signal value of the IR signal is smallest in the target area.

For example, the reference value β is the signal value of a lowest level of the IR signal detected according to light emission of ICG administered to the inside of the body. The reference value β is determined on the basis of the type of object 60, the excitation light intensity of the light source 100, and the density of ICG administered to the inside of the body. The reference value β is determined on the basis of the information at the time of imaging an object 60 including a known target area, and the determined reference value β is recorded in the memory 400.

The signal processing unit 40 (the fluorescence area determining unit 403) generates fluorescence area information on the basis of a result of the determination of the fluorescence area. The fluorescence area information includes the positional information of pixels determined to be included in the fluorescence area. The signal processing unit 40 (the fluorescence area determining unit 403) outputs the fluorescence area information to the IR signal processing unit 404.

As described above, the signal value of the IR signal generated by a pixel, on which light from an object not emitting fluorescence and having high reflectivity of the excitation light is incident, is large. When a fluorescence area is determined on the basis of IR signals corresponding to the entire imaged area of the object, there is a possibility that a pixel of which the signal value of the IR signal is large is erroneously determined as a fluorescence area in an area other than the target area. However, the signal processing unit 40 (the fluorescence area determining unit 403) determines a fluorescence area on the basis of IR signals of only the target area. Accordingly, the signal processing unit 40 (the fluorescence area determining unit 403) can determine a fluorescence area with high accuracy.

The signal processing unit 40 (the IR signal processing unit 404) performs an emphasis process of the signal value of the second image signal (IR signal) of each pixel corresponding to the fluorescence area. In this way, the signal processing unit 40 (IR signal processing unit 404) generates a fluorescence image signal. When the emphasis process is performed, the signal processing unit 40 (the IR signal processing unit 404) performs the following process. The signal processing unit 40 (the IR signal processing unit 404) performs the emphasis process by adding a predetermined value only to the signal value of the IR signal corresponding to the fluorescence area. In other words, the signal processing unit 40 (the IR signal processing unit 404) adds a predetermined value γ only to the signal value of the IR signal of each pixel corresponding to the fluorescence area. The predetermined value γ is set to be larger than zero and is set such that a maximum value of the IR signal after the addition is a value less than a saturated signal value. The predetermined value γ may be larger than the signal value of a lowest-level IR signal detected as ICG administered to the inside of the body emits light.

By adding the predetermined value γ only to the signal value of the IR signal corresponding to the fluorescence area, a difference between the signal value of the IR signal after the addition and the signal value of the IR signal corresponding to an area other than the fluorescence area increases. For this reason, IR signals corresponding to the fluorescence area are more emphasized.

The signal processing unit 40 (the IR signal processing unit 404) may perform the emphasis process by adding a value according to a signal value only to the signal value of the IR signal corresponding to the fluorescence area. In other words, the signal processing unit 40 (the IR signal processing unit 404) may perform the emphasis process by adding another value according to a signal value only to the signal value of the IR signal of each pixel corresponding to the fluorescence area. The added value is larger than “0” and is smaller than the maximum value (or the saturated signal value) of the IR signal. The signal processing unit 40 (the IR signal processing unit 404) adds a larger value to a larger signal value of the IR signal.

By adding a value corresponding to a signal value only to the signal value of an IR signal according to the fluorescence area, a difference between a signal value of the IR signal after the addition and an IR signal corresponding to an area other than the fluorescence area is increased. For this reason, an IR signal corresponding to the fluorescence area is more emphasized. By adding a larger value to a larger signal value of an IR signal, a difference between intensities of IR signals in the fluorescence area is increased.

The signal processing unit 40 (the IR signal processing unit 404) may perform the emphasis process by multiplying only the signal value of an IR signal corresponding to the fluorescence area by a predetermined value. In other words, the signal processing unit 40 (the IR signal processing unit 404) multiplies only the signal value of the IR signal of each pixel corresponding to the fluorescence area by a predetermined value γa. The predetermined value γa is set to be larger than “1” and is set such that a maximum value of the IR signal after the multiplication is a value smaller than the saturated signal value.

By multiplying only the signal value of an IR signal corresponding to the fluorescence area by the predetermined value γa, a difference between the signal value of the IR signal after the multiplication and the signal value of an IR signal corresponding to an area other than the fluorescence area is increased. For this reason, the IR signals corresponding to the fluorescence area are more emphasized.

The signal processing unit 40 (the IR signal processing unit 404) may perform the emphasis process by multiplying only the signal value of an IR signal corresponding to the fluorescence area by a value according to the signal value. In other words, the signal processing unit 40 (the IR signal processing unit 404) may perform the emphasis process by multiplying only the signal value of the IR signal of each pixel corresponding to the fluorescence area by a value that differs according to the signal value. The multiplier is set to be larger than “1” and is set such that a maximum value of the IR signal after the multiplication is a value smaller than the saturated signal value. The signal processing unit 40 (IR signal processing unit 404) multiplies a larger signal value of an IR signal by a larger value.

By multiplying only the signal value of an IR signal corresponding to the fluorescence area by a value according to the signal value, a difference between the signal value of the IR signal after the multiplication and the signal value of an IR signal corresponding to an area other than the fluorescence area is increased. For this reason, the IR signals corresponding to the fluorescence area are more emphasized. By multiplying a larger signal value of an IR signal by a larger value, a difference between the intensities of IR signals in the fluorescence area is further increased.

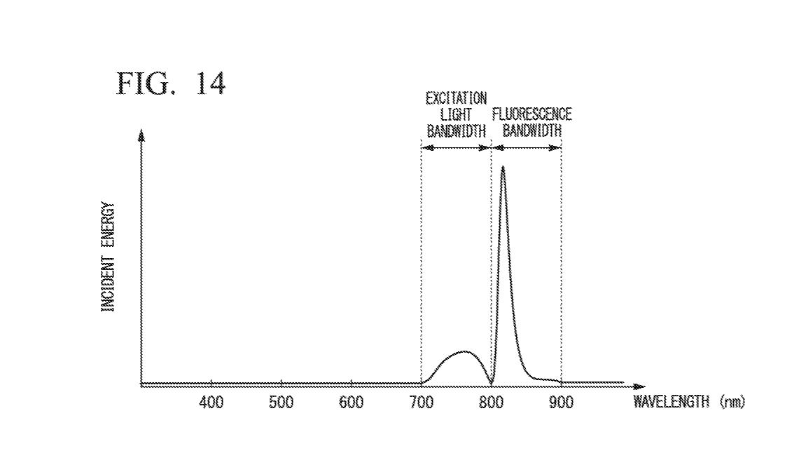

The signal processing unit 40 (the IR signal processing unit 404) may perform an emphasis process of the second image signal (IR signal) corresponding to the fluorescence area and perform a process of decreasing the second image signal (IR signal) corresponding to an area other than the fluorescence area. When the decreasing process is performed, the signal processing unit 40 (the IR signal processing unit 404) performs the following process. The signal processing unit 40 (the IR signal processing unit 404) performs the decreasing process by subtracting a predetermined value from only the signal values of IR signals corresponding to an area other than the fluorescence area. In other words, the signal processing unit 40 (the IR signal processing unit 404) subtracts a predetermined value γb from only the signal value of an IR signal of each pixel corresponding to an area other than the fluorescence area. The predetermined value γb is larger than “0” and is smaller than the maximum signal value of the IR signal based on the component of the excitation light shown in FIG. 14.

By subtracting the predetermined value γb from only the signal values of IR signals corresponding to an area other than the fluorescence area, a difference between the signal value of an IR signal after the subtraction and the signal value of an IR signal corresponding to a fluorescence area is increased. In this way, the IR signal corresponding to an area other than the fluorescence area is further decreased.

The signal processing unit 40 (the IR signal processing unit 404) may perform the decreasing process by multiplying only the signal values of IR signals corresponding to an area other than the fluorescence area by a value less than “1.” In other words, the signal processing unit 40 (the IR signal processing unit 404) may perform the decreasing process by multiplying only the signal value of the IR signal of each pixel corresponding to the area other than the fluorescence area by a value less than “1.” The value of the multiplier may be either a constant or a value that differs according to the signal value of the IR signal.

By multiplying only the signal values of IR signals corresponding to an area other than the fluorescence area by a value less than “1,” a difference between the signal value of an IR signal after the multiplication and the signal value of the IR signal corresponding to the fluorescence area is increased. In this way, an IR signal corresponding to an area other than the fluorescence area is further decreased.

The signal processing unit 40 (the IR signal processing unit 404) outputs a fluorescence image signal to the monitor 500. The fluorescence image signal includes IR signals corresponding to an area other than the fluorescence area and IR signals, corresponding to the fluorescence area, for which the emphasis process has been performed.

The imaging apparatus according to each aspect of the present invention may not include a configuration corresponding to at least one of the light source unit 10, the endoscope unit 20, the imaging lens 300, the dichroic mirror 301, the excitation light cutoff filter 302, the dichroic prism 304, and the display unit 50.

In the first embodiment, the signal processing unit 40 determines a target area of the object 60 on the basis of an R signal, a G signal, and a B signal. The signal processing unit 40 determines a fluorescence area on the basis of IR signals corresponding to the target area. The signal processing unit 40 performs an emphasis process of IR signals corresponding to the fluorescence area. For this reason, the endoscope apparatus 1a can generate a fluorescence image signal used for displaying a fluorescence image in which the fluorescence area is brightened more clearly.

The signal processing unit 40 performs addition or multiplication only for the signal values of IR signals corresponding to the fluorescence area. Accordingly, in a fluorescence image, the fluorescence area stands out more than other areas.

The endoscope apparatus 1a separately acquires an R signal, a G signal, a B signal, and an IR signal. For this reason, the endoscope apparatus 1a can acquire a visible light image and a fluorescence image having high resolution. In addition, the endoscope apparatus 1a can simultaneously perform imaging of visible light and imaging of infrared light.

The signal processing unit 40 determines a target area on the basis of the saturation and the hue of the R signal, the G signal, and the B signal of each pixel. In this way, a target area can be determined on the basis of the saturation and the hue.

### Second Embodiment

A second embodiment of the present invention will be described using the endoscope apparatus 1a shown in FIG. 1. Hereinafter, points different from those of the first embodiment will be described.

A signal processing unit 40 (target area determining unit 402) calculates an area determination coefficient of each pixel according to a degree of correlation between signal values of the first image signals (an R signal, a G signal, and a B signal) of each pixel and a reference value. The reference value corresponds to a value expected as the signal value of a first image signal corresponding to the target area. The signal processing unit 40 (the target area determining unit 402) determines a target area on the basis of the area determination coefficient calculated for each pixel.

The area determination coefficient represents the certainty of each pixel being in a target area. The signal processing unit 40 (the target area determining unit 402) determines a possibility that each pixel belongs to a target area on the basis of the area determination coefficient. Accordingly, the signal processing unit 40 (the target area determining unit 402) can determine a target area according to the degree of certainty of being the target area.

The signal processing unit 40 (the target area determining unit 402) multiplies the signal value of the second image signal (IR signal) of each pixel for which the emphasis process has been performed by the area determination coefficient of each pixel.

The area determination coefficient of each pixel of a case in which each pixel of the first image signal is included in the target area is larger than the area determination coefficient of each pixel of a case in which each pixel of the first image signal is not included in the target area. For this reason, by multiplying the signal value of the second image signal by the area determination coefficient, the ratio of the signal values of pixels included in the target area and the fluorescence area to the signal values of pixels not included in the target area is increased. As a result, in a fluorescence image, a fluorescence area stands out more than other areas.

Details of the process performed by the signal processing unit 40 (the target area determining unit 402) will be described. When an area determination coefficient is calculated, the signal processing unit 40 (the target area determining unit 402) performs the following process. The signal processing unit 40 (the target area determining unit 402) reads a reference value from a memory 400. The signal processing unit 40 (the target area determining unit 402) compares the reference value recorded in the memory 400 with RGB information generated by an RGB signal processing unit 401. The signal processing unit 40 (the target area determining unit 402) calculates a degree of correlation on the basis of a result of the comparison. The signal processing unit 40 (the target area determining unit 402) calculates an area determination coefficient on the basis of the calculated degree of correlation.

When the RGB information is ratios between an R signal, a G signal, and a B signal, the RGB information generated by the RGB signal processing unit 401 includes a ratio X3 between the signal values of the R signal and the G signal and a ratio Y3 between the signal values of the R signal and the B signal of each pixel. The reference value recorded in the memory 400 is a ratio X5 between the signal values of the R signal and the G signal in the target area and a ratio Y5 of the signal values of the R signal and the B signal in the target area. As described above, the ratio between the signal values of the R signal and the G signal in the target area is in the range of X1 to X2. Here, X5 is a representative value of the range of X1 to X2. As described above, the ratio between the signal values of the B signal and the G signal in the target area is in the range of Y1 to Y2. Here, Y5 is a representative value of the range of Y1 to Y2.

The signal processing unit 40 (the target area determining unit 402) compares the combination of the ratio X3 and the ratio Y3 of each pixel with the combination of the ratios X5 and Y5 that are the reference values and calculates a degree of correlation. For example, the signal processing unit 40 (the target area determining unit 402) calculates a Euclidean distance between (X3, Y3) and (X5, Y5). The calculated Euclidean distance represents a degree of correlation between the signal values of the R signal, the G signal, and the 13 signal of each pixel and the reference values. When the Euclidean distance is short, the degree of correlation is high. On the other hand, when the Euclidean distance is long, the degree of correlation is low.

The signal processing unit 40 (the target area determining unit 402) calculates an area determination coefficient of each pixel on the basis of the degree of correlation of each pixel. For example, the area determination coefficient of each pixel is a value in the range of “0” to “1.” When the degree of correlation is high, in other words, when there is a high possibility that each pixel is included in the target area, the area determination coefficient is close to “1.” On the other hand, when the degree of correlation is low, in other words, when there is a high possibility that each pixel is not included in the target area, the area determination coefficient is close to “0.” In other words, the area determination coefficient has a weighting factor according to the degree of correlation.

The signal processing unit 40 (the target area determining unit 402) compares the area determination coefficient of each pixel with a reference value δ. Here, the reference value δ is a value larger than “0” and smaller than “1.” In this way, the signal processing unit 40 (the target area determining unit 402) determines whether or not each pixel is included in the target area.

When the area determination coefficient of each pixel is the reference value δ or more, the signal processing unit 40 (the target area determining unit 402) determines that the pixel that is the determination target is included in the target area. On the other hand, when the area determination coefficient of each pixel is less than the reference value δ, the signal processing unit 40 (the target area determining unit 402) determines that the pixel that is the determination target is not included in the target area.

For example, the ratio X5 and the ratio Y5 that are the reference values are determined as below. The signal processing unit 40 (the RGB signal processing unit 401) can acquire a representative spectrum distribution of visible light that is reflected by the target area and is incident on the image sensor on the basis of known information. The known information includes the spectrum distribution of light emitted by a light source 100, the spectral transmittance depending on the optical system of the endoscope apparatus 1a, and the spectral reflection characteristics of the target area. The signal processing unit 40 (the RGB signal processing unit 401) calculates the representative ratios X5 and Y5 in the target area on the basis of a representative spectrum distribution of visible light. The ratios X5 and Y5 that have been calculated are recorded in the memory 400. The signal processing unit 40 (the RGB signal processing unit 401) may calculate the representative ratios X5 and Y5 in the target area on the basis of the R signal, the G signal, and the B signal generated when the object 60 including the known target area is imaged.

For example, the reference value δ is determined as below. The ratio X5 and the ratio Y5 are representative values in the target area. However, due to noise generated in the image sensor, unevenness of light emitted by the light source 100, and the like, the ratio X3 and the ratio X5 between the signal values of the R signal and the G signal are not necessarily the same in the target area. Similarly, the ratio Y3 and the ratio Y5 between the signal values of the R signal and the B signal are not necessarily the same in the target area. In other words, the ratio X3 and the ratio Y3 detected in the target area have variations. Even when the ratio X3 and the ratio Y3 have variations in the target area, a reference value δ for determining that most pixels corresponding to the target area are in the target area is determined.

For example, the signal processing unit 40 (the RGB signal processing unit 401) calculates the ratio X3 and the ratio Y3 of each pixel of the target area on the basis of an R signal, a G signal, and a B signal generated when an object 60 including a known target area is imaged. The signal processing unit 40 (the RGB signal processing unit 401) calculates a degree of correlation between the ratio X3 and the ratio Y3 of each pixel of the target area and the ratio X5 and the ratio Y5 that are reference values. The signal processing unit 40 (the RGB signal processing unit 401) determines a reference value δ on the basis of the distribution of the degree of correlation of each pixel.

When the RGB information is the saturation and the hue, the signal processing unit 40 (the target area determining unit 402) compares the combination of the saturation and the hue of each pixel and the combination of the saturation and the hue that are reference values and calculates a degree of correlation thereof. The calculation of an area determination coefficient based on the degree of correlation and the determination of a target area based on the area determination coefficient are similar to the processes described above.

The signal processing unit 40 (the fluorescence area determining unit 403) determines a fluorescence area by using a method similar to that according to the first embodiment. The signal processing unit 40 (the fluorescence area determining unit 403) generates fluorescence area information on the basis of a result of the determination of the fluorescence area. The fluorescence area information includes positional information of pixels determined to be included in the fluorescence area. The signal processing unit 40 (the fluorescence area determining unit 403) outputs the fluorescence area information and the area determination coefficient of each pixel to the IR signal processing unit 404.

The signal processing unit 40 (IR signal processing unit 404) performs the emphasis process according to the first embodiment for a second image signal (IR signal). In other words, the signal processing unit 40 (IR signal processing unit 404) performs the emphasis process by performing addition or multiplication of a predetermined value only for the signal values of IR signals corresponding to the fluorescence area. The signal processing unit 40 (IR signal processing unit 404) may perform the emphasis process by performing addition or multiplication of a value according to signal values of IR signals corresponding to the fluorescence area only for the signal values.

In addition, the signal processing unit 40 (IR signal processing unit 404) performs the following process. The signal processing unit 40 (the target area determining unit 402) multiplies the signal value of the IR signal of each pixel for which the emphasis process has been performed by the area determination coefficient of each pixel. The multiplication of the signal value of the IR signal and the area determination coefficient corresponding to the same pixel is performed.

As described above, the area determination coefficient of each pixel is a value in the range of “0” to “1.” When there is a high possibility that each pixel is included in the target area, the area determination coefficient is close to “1.” On the other hand, when there is a low possibility that each pixel is included in the target area, the area determination coefficient is close to “0.” For example, a ratio Pr1 between the signal value Sir1 of an IR signal of a pixel P1 corresponding to the target area and the fluorescence area and the signal value Sir2 of an IR signal of a pixel P2 corresponding to an area other than the target area is represented in Equation (1).

Pr1=Sir1/Sir2 (1)

The area determination coefficient of the pixel P1 is a1, and the area determination coefficient of the pixel P2 is a2. After the signal values of IR signals are multiplied by the area determination coefficients, a ratio Pr2 between the signal value Sir1′ of an IR signal of the pixel P1 corresponding to the target area and the fluorescence area and the signal value Sir2′ of an IR signal of the pixel P2 corresponding to an area other than the target area is represented in Equation (2).

Pr2=Sir1′/Sir2′=(a1×Sir1)/(a2×Sir2) (2)

The area determination coefficient a1 is larger than the area determination coefficient a2. For this reason, the ratio Pr2 is higher than the ratio Pr1. In other words, by multiplying the signal value of the IR signal by the area determination coefficient, in a fluorescence image, the fluorescence area stands out more than the other areas.

The signal processing unit 40 (IR signal processing unit 404) outputs a fluorescence image signal to the monitor 500. The fluorescence image signal includes IR signals corresponding to an area other than the fluorescence area and IR signals, corresponding to the fluorescence area, for which the emphasis process and the multiplication of the area determination coefficient have been performed.

The signal processing unit 40 (IR signal processing unit 404) may perform the emphasis process and the decreasing process according to the first embodiment. In the second embodiment, the multiplication of the signal value of the IR signal of each pixel by the area determination coefficient of each pixel is not essential.

Regarding points other than those described above, the operation of the endoscope apparatus 1a according to the second embodiment is similar to the operation of the endoscope apparatus 1a according to the first embodiment.

In the second embodiment, the endoscope apparatus 1a can generate a fluorescence image signal for displaying a fluorescence image in which a fluorescence area is brightened more clearly.

The signal processing unit 40 calculates an area determination coefficient of each pixel according to the degree of correlation between the signal values of the R signal, the G signal, and the B signal of each pixel and the reference values. The signal processing unit 40 determines a target area on the basis of the area determination coefficient. In this way, the signal processing unit 40 determines a target area according to the certainty of being a target area.

The signal processing unit 40 multiplies the signal value of the IR signal of each pixel for which the emphasis process has been performed by the area determination coefficient of the pixel. In this way, the endoscope apparatus 1a can generate a fluorescence image signal for displaying a fluorescence image in which the fluorescence area is brightened more clearly.

### First Modified Example

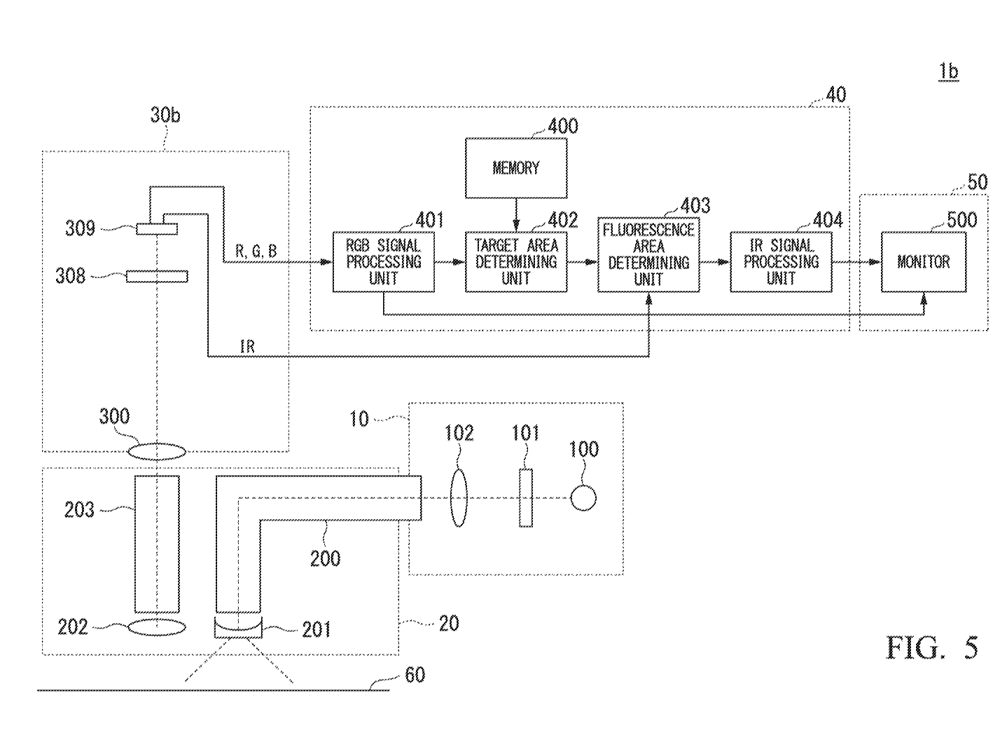

FIG. 5 shows the configuration of an endoscope apparatus 1b according to a first modified example of the first and second embodiments of the present invention. As shown in FIG. 5, the endoscope apparatus 1b includes a light source unit 10, an endoscope unit 20, a camera head 30b (imaging unit), signal processing unit 40, and a display unit 50. FIG. 5 shows schematic configurations of the light source unit 10, the endoscope unit 20, and the camera head 30b.

In the configuration shown in FIG. 5, points different from those of the configuration shown in FIG. 1 will be described. The camera head 30b includes an imaging lens 300, an excitation light cutoff filter 308, and an image sensor 309 (a visible light imaging unit and a fluorescence imaging unit). The imaging lens 300 is the same as the imaging lens 300 shown in FIG. 1.

First light that has been transmitted through the imaging lens 300, in other words, the first light from an object 60 is incident on the excitation light cutoff filter 308. The light incident on the excitation light cutoff filter 308 includes visible light and infrared light. The visible light includes red light, green light, and blue light. The infrared light includes excitation light and fluorescence. The excitation light cutoff filter 308 blocks the excitation light and transmits the fluorescence and the visible light.

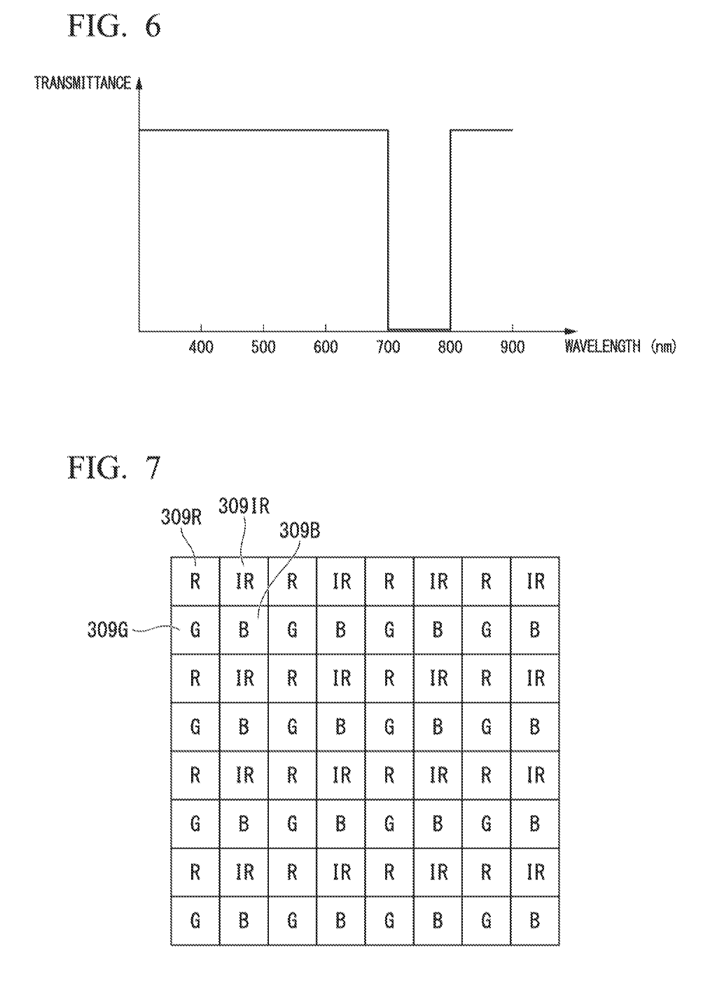

FIG. 6 shows the transmission characteristics of the excitation light cutoff filter 308. In a graph shown in FIG. 6, the horizontal axis indicates the wavelength, and the vertical axis indicates the transmittance. The excitation light cutoff filter 308 blocks light of a wavelength band having wavelengths having about 700 nm to 800 nm. On the other hand, the excitation light cutoff filter 308 transmits light of a wavelength band having wavelengths to be less than about 700 nm and light of a wavelength band having wavelengths to be about 800 nm or more. The wavelength band of the light blocked by the excitation light cutoff filter 308 includes the wavelength band of the excitation light. The wavelength band of the light transmitted by the excitation light cutoff filter 308 includes the wavelength band of the visible light and the wavelength band of the fluorescence. The blocking characteristics of the excitation light cutoff filter 308 for the excitation light is not perfect. The excitation light cutoff filter 308 blocks a part of light of the wavelength band of the excitation light and transmits the remaining light of the wavelength band of the excitation light, the fluorescence, and the visible light.

The excitation light and the fluorescence that have been transmitted through the excitation light cutoff filter 308 are incident on the image sensor 309. The image sensor 309 generates an R signal (first image signal) on the basis of red light, a G signal (first image signal) on the basis of green light, and a B signal (first image signal) on the basis of blue light. IN addition, the image sensor 309 generates an IR signal (second image signal) on the basis of the excitation light and the fluorescence.

Regarding points other than those described above, the configuration shown in FIG. 5 is similar to the configuration shown in FIG. 1.

FIG. 7 shows the pixel arrangement of the image sensor 309. The image sensor 309 includes a plurality of pixels 309R, a plurality of pixels 309G, a plurality of pixels 309B, and a plurality of pixels 309IR. The plurality of pixels 309R, the plurality of pixels 309G, the plurality of pixels 309B, and the plurality of pixels 309IR are arranged in a matrix pattern. In FIG. 7, signs of one pixel 309R, one pixel 309G, one pixel 309B, and one pixel 309IR are representatively shown. The one pixel 309R, the one pixel 309G, the one pixel 309B, and the one pixel 309IR configure a unit array. In the pixel arrangement shown in FIG. 7, a plurality of unit arrays are periodically arranged in a two-dimensional shape.

Filters transmitting red light are arranged on the surfaces of the plurality of pixels 309R. Filters transmitting green light are arranged on the surfaces of the plurality of pixels 309G. Filters transmitting blue light are arranged on the surfaces of the plurality of pixels 309B. Filters transmitting fluorescence are arranged on the surfaces of the plurality of pixels 309IR. The plurality of pixels 309R generate R signals on the basis of the red light. The plurality of pixels 309G generate G signals on the basis of the green light. The plurality of pixels 309B generate B signals on the basis of the blue light. The plurality of pixels 309IR generate IR signals on the basis of the fluorescence. Thus, the plurality of pixels 309R, the plurality of pixels 309G and the plurality of pixels 309B configure a visible light imaging unit. The plurality of pixels 309IR configure a fluorescence imaging unit.

### Second Modified Example

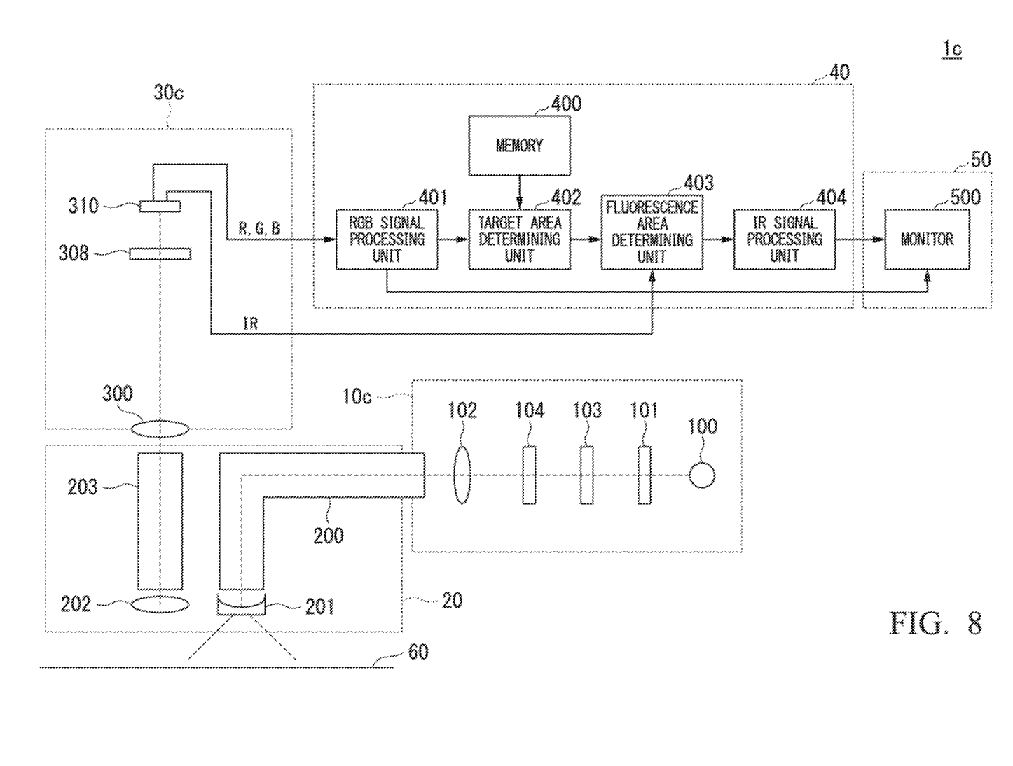

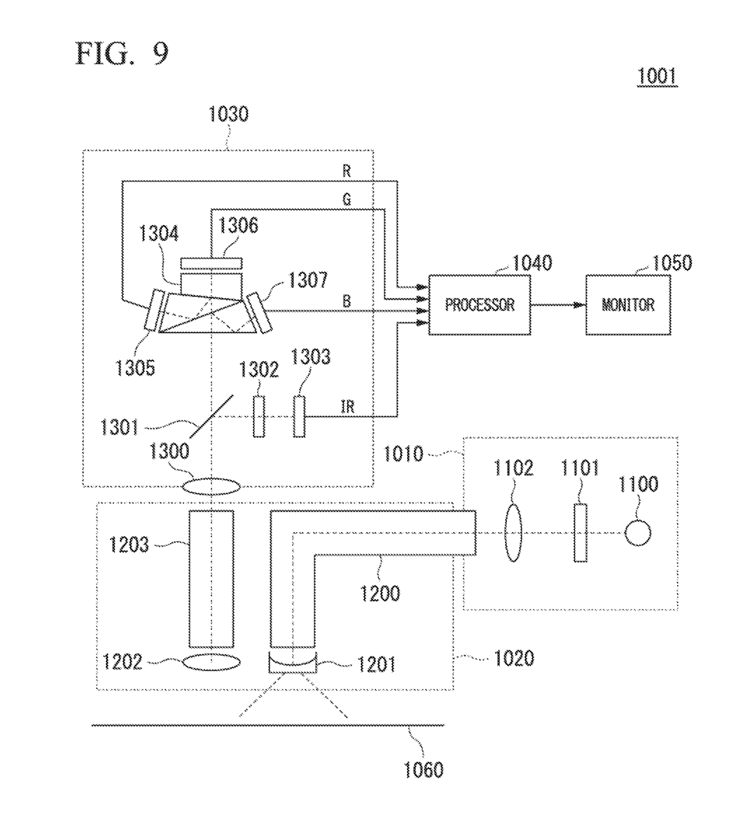

FIG. 8 shows the configuration of an endoscope apparatus 1c according to a second modified example of the first and second embodiments of the present invention. As shown in FIG. 8, the endoscope apparatus 1c includes a light source unit 10c, an endoscope unit 20, a camera head 30c (imaging unit), signal processing unit 40, and a display unit 50. FIG. 8 shows schematic configurations of the light source unit 10c, the endoscope unit 20, and the camera head 30c.

In the configuration shown in FIG. 8, points different from those of the configuration shown in FIG. 5 will be described. The light source unit 10c includes a light source 100, a band pass filter 101, a condenser lens 102, a bandlimiting filter 103, and an RGB rotation filter 104. The light source 100 is the same as the light source 100 shown in FIG. 1. The band pass filter 101 is the same as the band pass filter 101 shown in FIG. 1. The condenser lens 102 is the same as the condenser lens 102 shown in FIG. 1.

Visible light and excitation light that are transmitted through the band pass filter 101 are incident on the bandlimiting filter 103. The bandlimiting filter 103 includes a first filter and a second filter. The first filter transmits only the visible light. The second filter transmits only the excitation light. The bandlimiting filter 103 is a rotation-type filter. One of the first filter and the second filter is arranged in an optical path. When imaging of visible light is performed, the first filter is arranged in the optical path. The bandlimiting filter 103 transmits the visible light. When imaging of fluorescence is performed, the second filter is arranged in the optical path. The bandlimiting filter 103 transmits the excitation light.

The light that has been passed through the bandlimiting filter 103 is incident on the RGB rotation filter 104. The RGB rotation filter 104 includes a third filter, a fourth filter, and a fifth filter. The third filter blocks the green light and the blue light and transmits the red light and the excitation light. The fourth filter blocks the red light and the blue light and transmits the green light and the excitation light. The fifth filter blocks the red light and the green light and transmits the blue light and the excitation light. The RGB rotation filter 104 is a rotation-type filter. The third filter, the fourth filter, and the fifth filter are sequentially arranged in the optical path. When the imaging of the visible light is performed, the RGB rotation filter 104 sequentially transmits the red light, the green light, and the blue light. On the other hand, when the imaging of fluorescence is performed, the RGB rotation filter 104 transmits the excitation light.

The camera head 30c includes an imaging lens 300, an excitation light cutoff filter 308, and an image sensor 310 (a visible light imaging unit and a fluorescence imaging unit). The imaging lens 300 is the same as the imaging lens 300 shown in FIG. 1. The excitation light cutoff filter 308 is the same as the excitation light cutoff filter 308 shown in FIG. 8.

The image sensor 310 has sensitivity for the visible light and the fluorescence. When the imaging of the visible light is performed, the red light, the green light and the blue light are sequentially transmitted through the excitation light cutoff filter 308. The image sensor 310 generates an R signal on the basis of the red light, a G signal on the basis of the green light, and a B signal on the basis of the blue light. When the imaging of the fluorescence is performed, the excitation light and the fluorescence are transmitted through the excitation light cutoff filter 308. The image sensor 310 generates an IR signal on the basis of the excitation light and the fluorescence.

As described above, the image sensor 310 can generate the R signal, the G signal, the B signal, and the IR signal at different timings.

Regarding points other than those described above, the configuration shown in FIG. 8 is similar to the configuration shown in FIG. 5.

As above, while the preferred embodiments of the present invention have been described, the present invention is not limited to these embodiments and the modified examples thereof. An addition, omission, substitution, and any other changes of the configurations can be made in a range not departing from the concept of the present invention. In addition, the present invention is not limited to the description presented above but is limited only by the scope of the attached claims.

What is claimed is:

1. An imaging apparatus, comprising:
an imaging unit configured to generate a first image signal on the basis of visible light from an object and generate a second image signal on the basis of excitation light and fluorescence from the object; and
a signal processing unit configured to generate a fluorescence image signal corresponding to the fluorescence on the basis of the first image signal and the second image signal,
wherein the signal processing unit determines a target area of the object on the basis of the first image signal,
the signal processing unit determines a fluorescence area on the basis of the second image signal corresponding to the target area, the fluorescence area generating the fluorescence in the object, and
the signal processing unit performs an emphasis process of the second image signal corresponding to the fluorescence area.

2. The imaging apparatus according to claim 1, wherein the signal processing unit performs the emphasis process by performing addition or multiplication of a predetermined value only for a signal value of the second image signal corresponding to the fluorescence area.

3. The imaging apparatus according to claim 1, wherein the signal processing unit performs the emphasis process by performing addition or multiplication of a value according to a signal value of the second image signal corresponding to the fluorescence area only for the signal value.

4. The imaging apparatus according to claim 1,
wherein the signal processing unit calculates an area determination coefficient of each pixel according to a degree of correlation between a signal value of the first image signal of each pixel and a reference value, the reference value corresponding to a value expected as a signal value of the first image signal corresponding to the target area, and
the signal processing unit determines the target area on the basis of the area determination coefficient.

5. The imaging apparatus according to claim 4, wherein the signal processing unit multiplies a signal value of the second image signal of each pixel for which the emphasis process is performed by the area determination coefficient of each pixel.

6. The imaging apparatus according to claim 1,
wherein the imaging unit includes:
a dichroic mirror configured to split first light from the object into second light and third light, the first light including the visible light, the excitation light, and the fluorescence, the second light including the visible light, and the third light including the excitation light and the fluorescence;
a visible light imaging unit, on which the second light is incident, configured to generate the first image signal;
an excitation light cutoff filter, on which the third light is incident, having first transmittance for the fluorescence and second transmittance for the excitation light, the first transmittance being higher than the second transmittance; and
a fluorescence imaging unit, on which the third light transmitted through the excitation light cutoff filter is incident, configured to generate the second image signal, and
the visible light imaging unit and the fluorescence imaging unit are connected to the signal processing unit.

7. The imaging apparatus according to claim 1,
wherein the signal processing unit includes:
a memory in which object characteristic information representing characteristics of the object is recorded, the object characteristic information generated on the basis of the first image signal of the object; and
a target area determining unit configured to determine the target area on the basis of the object characteristic information recorded in the memory and the first image signal.

8. The imaging apparatus according to claim 1,
wherein the signal processing unit calculates saturation and hue of each pixel on the basis of a signal value of the first image signal of each pixel, and
the signal processing unit determines the target area on the basis of the saturation and the hue of each pixel.
